(19) United States
(12) Patent Application Publication
(10) Pub. No.: US 2014/0276548 A1
(43) Pub. Date: Sep. 18, 2014

(54) Fluid Bolus Delivery

(71) Applicant: CAREFUSION 303, INC., San Diego, CA (US)

(72) Inventors: Stephen Bollish, San Diego, CA (US); Jesse J. Guerra, San Diego, CA (US)

(73) Assignee: CareFusion 303, Inc., San Diego, CA (US)

(21) Appl. No.: 13/831,407

(22) Filed: Mar. 14, 2013

(51) Int. Cl. A61M 5/172

(52) U.S. Cl. CPC A61M 5/1723; A61M 5/172
USPC 604/503; 604/506; 604/151; 604/66

(57) ABSTRACT

The subject matter described herein relates to controlling and automating a delivery of one or boluses of a fluid medication to a patient. A user interface can display lists of parameters, such as a list of types of fluid in the fluid medication, a list of modes for delivery of the one or more boluses, a list of flow rates of each bolus delivery, a list of durations corresponding to each bolus, a list of initiation times of each bolus, and a list of initiation events for each bolus. In response, a clinician can select and/or specify parameters for the one or more boluses. Subsequently, the infusion device can deliver the one or more boluses based on the selected or specified parameters. Related apparatus, systems, techniques and articles are also described.

### TECHNICAL FIELD

The subject matter described herein relates to controlling and automating a provision of one or boluses of a fluid medication to a patient.

### BACKGROUND

Infusion pumps are known to infuse medications into a body of a patient. A clinician can program the infusion pump to infuse medication at a first flow rate. The clinician may need to infuse a bolus of medication at a particular time in future. Conventionally, at this particular time, the clinician goes to the infusion pump and reprograms the infusion pump to infuse medication at a second flow rate, which is higher than the first flow rate. When the bolus has been infused into the patient, the clinician goes again to the infusion pump and reprograms the infusion pump to infuse medication at the first flow rate. If the clinician needs to infuse multiple boluses at different times, the clinician needs to go multiple times to the infusion pump and reprogram the infusion pump to change the flow rates of infusion. This repetitive behavior of going to the pump and reprogramming the pump can be inconvenient for the clinician.

### SUMMARY

The current subject matter relates to a medical device (for example, an infusion pump) that can control and automate the delivery of one or more boluses of a fluid medication to a patient. A user interface can display lists of parameters, such as a list of types of fluid in the fluid medication, a list of modes for delivery of the one or more boluses, a initial flow rates of each bolus delivery, an at least an initial duration corresponding to each bolus, a list of initiation times of each bolus, and a list of initiation events for each bolus. In response, a clinician can select or specify parameters for the one or more boluses. Subsequently, the infusion device can deliver the one or more boluses based on the selected or specified parameters. In some variations, the infusion device, prior to the delivery of the one or more boluses, infuses the fluid medication at a first rate. The infusion device also, immediately subsequent to the delivery of the one or more boluses, automatically and without human intervention, infuses the fluid medication at the first rate (which is different than a flow rate for the one or more boluses).

In one aspect, in a graphical user interface, a list of possible flow rates of one or more boluses of a fluid medication to be delivered to a patient and a list of possible durations for the one or more boluses, and a list of possible initiation times for the one or more boluses can be displayed. From the graphical user interface and by a controller connected to the graphical user interface, selections can be received. The selections can include selections of: one or more flow rates from the list of possible flow rates for the one or more boluses, one or more durations from the list of possible durations for the one or more boluses, and one or more initiation times from the list of possible initiation times for the one or more boluses. The selections can be performed via the graphical user interface by a clinician. The controller can actuate an infusion device that can deliver, based on the selections, the one or more boluses to the patient.

The infusion device can infuse, prior to the delivery of the one or more boluses, the fluid medication at a first rate. The infusion device can infuse, immediately subsequent to the delivery of the one or more boluses automatically and without human intervention, the fluid medication at the first rate. The first rate can be different from a flow rate for the one or more boluses.

A list of possible types of fluid in the fluid medication can be displayed in the graphical user interface. A selection of a type of fluid to be used in the fluid medication can be received from the graphical user interface and by the controller. Further, a list of possible modes for delivery of the one or more boluses can be displayed in the user interface. The modes can include a single bolus mode and a multiple bolus mode. The single bolus mode can characterize that a single bolus of fluid medication is to be delivered. The multiple bolus mode can characterize that two or more boluses are to be delivered. A selection of a mode to be used for delivering the one or more boluses can be received by the controller from the graphical user interface.

In the graphical user interface, a list of possible initiation events for each bolus can be displayed. The controller can receive a selection of an initiation event from the graphical user interface. The infusion device can deliver at least one of the one or more boluses when the initiation event occurs. The initiation events can include one or more of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

Further, in the graphical user interface, a graphical waveform can be displayed. The graphical waveform can characterize the selections of the one or more flow rates, the one or more durations, and the one or more initiation times.

A notification device or an alarm device can be actuated by the controller to generate one or more alarms when a volume of a delivered bolus of the fluid medication either drops below a lower limit threshold or exceeds an upper limit threshold.

In another aspect, a system is described that includes a user interface device, a controller, and an infusion device. The user interface device can display a list of possible flow rates of one or more boluses of a fluid medication to be delivered to a patient, a list of possible durations for the one or more boluses, and a list of possible initiation times for the one or more boluses. The user interface device can receive selections of one or more flow rates of corresponding one or more boluses, one or more durations of the corresponding one or more boluses, and one or more initiation times of the corresponding one or more boluses. The controller can receive the selections from the user interface device. The infusion device can receive at least one control signal from the controller based on the selections. The at least one control signal can actuate the infusion device to deliver the one or more boluses to the patient.

The infusion device can infuse, prior to the delivery of the one or more boluses, the fluid medication at a first rate. The infusion device can infuse, immediately subsequent to the delivery of the one or more boluses automatically and without human intervention, the fluid medication at the first rate. The first rate can be different than a flow rate for the one or more boluses.

The user interface device can display a list of possible types of fluid in the fluid medication. Further, the user interface can display a list of possible modes for delivery of the one or more boluses. The modes can include a single bolus mode and a multiple bolus mode. The single bolus mode can characterize that a single bolus of fluid medication is to be delivered. The multiple bolus mode can characterize that a plurality of boluses are to be delivered. Furthermore, the user interface device can further display a list of possible initiation events for each bolus.

The user interface device can receive a selection of a type of fluid to be used in the fluid medication. Further, the user interface device can receive a selection of a mode to be used for delivering the one or more boluses. Moreover, the user interface device can receive a selection of an initiation event. The controller can actuate the infusion device when the initiation event occurs so as to deliver at least one of the one or more boluses. The initiation event can include at least one of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

The user interface device can include a graphical display area that displays a graphical waveform characterizing the selections of the one or more flow rates, the one or more durations, the one or more initiation times, the type of fluid, the mode, and the initiation event.

The system can further include an alarm device that can generate one or more alarms when a volume of a delivered bolus of the fluid medication either drops below a lower limit threshold or exceeds an upper limit threshold. The alarm can include a popup message displayed on the graphical user interface device, a loud sound, a short messaging service notification, an email, and a social network message.

In yet another aspect, a list of possible flow rates of a bolus of a fluid medication to be delivered to a patient and a list of possible durations for the bolus are displayed in a graphical user interface of a user interface device. A controller connected to the graphical user interface can receive selections from the graphical user interface. The selections can include selections of a flow rate of the bolus from the list of possible flow rates, and a duration of the bolus from the list of possible durations, the selections being performed on the graphical user interface by a clinician. The controller can actuate an infusion device that can deliver, based on the selections, the bolus to the patient.

The infusion device can infuse, prior to the delivery of the bolus, the fluid medication at a first rate. The first rate can be different than a flow rate for the one or more boluses. The infusion device can infuse, immediately subsequent to the delivery of the bolus automatically and without human intervention, the fluid medication at the first rate.

In the graphical user interface, a list of possible types of fluid in the fluid medication can be displayed. From the graphical user interface and by the controller, a selection of a type of fluid to be used in the fluid medication can be received.

In the graphical user interface, a list of possible initiation events for the bolus can be received from the clinician. The controller can receive a selection of an initiation event from the graphical user interface. The infusion device can deliver the bolus when the initiation event occurs. The initiation events can include one or more of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

A graphical waveform characterizing the selections of the flow rate and the duration can be displayed in the graphical user interface. Further, the controller can actuate an alarm device that can generate one or more alarms when a volume of a delivered bolus of the fluid medication either drops below a lower limit threshold or exceeds an upper limit threshold.

Computer program products are also described that comprise non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many advantages. For example, parameters (for example, flow rate, volume, duration, initiation time, initiation event, and/or other parameters) associated with a delivery of one or more medication boluses to a patient can be specified and programmed in advance of the delivery of the boluses. Thus, the clinician may not be required to be present for every bolus delivery, thereby saving time of the clinician.

Further, a user interface is provided for a clinician to specify parameters associated with the delivery of one or more boluses. This user interface is user friendly, and is easy to use and navigate. Further, this user interface can be implemented on any computing device, such as a medical device, a computer, a tablet computer, a cellular phone, and other devices, thereby providing accessing convenience to the clinician. Moreover, the user interface can include a graphical display area showing a waveform characterizing a foreseen bolus delivery, thereby reducing a likelihood of error by a clinician while selecting or specifying the parameters associated with the one or more boluses.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating a medical device that provides one or more boluses of a fluid medication to a patient;

FIG. 2 is a diagram illustrating a waveform of a bolus of fluid medication delivered by the infusion device to the patient;

FIG. 3 is a diagram illustrating a waveform of multiple boluses of fluid medication delivered by the infusion device to the patient;

FIG. 4 is a diagram illustrating an infusion pump, which is an example of the medical device;

FIG. 5 is a diagram illustrating a user interface wirelessly connected with an infusion device that can deliver one or more boluses to a patient;

FIG. 6 is a diagram illustrating a user interface connected with an infusion device that can deliver one or more boluses to a patient;

FIG. 7 is a diagram illustrating an example of a user interface;

FIG. 8 is a flow diagram illustrating an automatic delivery of one or more boluses to a patient based on a prior selection or specification of bolus parameters by a clinician; and

FIG. 9 is a system diagram illustrating a computing landscape within a healthcare environment.

Like reference symbols in the various drawings indicate like elements.

### DETAILED DESCRIPTION

FIG. 1 is a diagram 100 illustrating a medical device 102 that provides one or more boluses of a fluid medication to a patient 104. The medical device 102 can include a user interface 106, a controller 108, and an infusion device 110. A clinician can use the user interface 106 to select or specify parameters for administering the fluid medication. These parameters can include a specification or selection of type of fluid medication, mode (for example, single bolus mode or multiple bolus mode) of provision of the fluid medication, flow rate (which can be measured in milliliters per kilogram per hour) corresponding to each bolus of fluid medication, duration corresponding to each bolus of fluid medication, time or event for initiation of each bolus of fluid medication, and the like. After the clinician inputs the parameters on the user interface 106, the user interface 106 can send the parameters to the controller 108. The controller 108 can then actuate the infusion device 110 to automatically provide the one or more boluses to the patient 104 in accordance with the parameters.

The fluid medication, for example, can include one or more of: Normosol-R, Plasma-Lyte-A, lactated Ringer's solution, 0.9% normal saline solution, fluids with higher osmolality than the blood cells and plasma, 5% dextrose in water, 0.45% sodium chloride, hetastarch, pentastarch, dextran, and hemoglobin glutamer-200 (Oxyglobin—Biopure), and any other suitable fluid medication. The clinician can use a drug library to specify the fluid medication and the associated parameters based on a type (for example, human being or animal) of the patient 104 and an age (for example, neonate, child, adult, old individual) of the patient 104. The drug library can characterize a mapping of fluid medications, types of patients, flow rate of fluid medication, upper threshold and lower threshold of the flow rate, duration of provision of medication, and flow rate and duration of one or more boluses.

The clinician can be a doctor and/or a nurse. In some variations, the clinician can be a pharmacist, an assistant or associate in a hospital or laboratory, a pharmacist, a psychiatrist, a psychologist, and/or any other authorized individual.

The user interface 106 can be an interactive graphic user interface that can receive data from the clinician and can display data to the clinician. In one implementation, the user interface 106 can be an interactive touch screen device. The clinician can input data to the user interface 106 using at least one of: a keypad, a mouse, a joystick, one or more touchscreen buttons on the user interface 106, a microphone, and other modes of inputting data.

The controller 108 can include one or more of: one or more microcontrollers, one or more processors, one or more computers, one or more servers, and the like.

The infusion device 110 can be a mechanical device that can deliver the medication to a body of the patient. The movements of the infusion device can be controlled by the controller 108. In some implementations, the infusion device 110 can include a plunger and a syringe. The controller 108 can turn a screw that can push on the plunger in accordance with parameters input by the clinician. In some implementations, the controller 108 can be embedded within the infusion device 110.

In some implementations, the medical device 102 can further include an notification device (for example, an alarm device) that can warn the clinician by generating one or more alarms when a volume or duration of a delivered programmed bolus either drops below a lower-permissible-limit threshold or exceeds an upper-permissible-limit threshold. The lower-permissible-limit threshold and the upper-permissible-limit threshold can be based on at least one of: the type of fluid in the medication, age of the patient, type of the patient (for example, human being, or animal), and the like. The alarm can be an alert message that can pop up on the user interface 106. In alternate implementations, the alarm can be one or more of: a loud sound that can have one or more tones based on the volume dropped or exceeded, a short messaging service notification, an email, and a social network message.

FIG. 2 is a diagram 200 illustrating a waveform of a bolus of fluid medication delivered by the infusion device 110 to the patient 104. Until time T1, the infusion device 110 can provide a flow rate R1 of a first medication to the patient 104. In some implementations, the value of flow rate R1 can be zero such that the first medication is not provided. From time T1 to time T2 (both times inclusive), the infusion device 110 can provide a bolus of a second medication having a flow rate R2 in addition to providing the first medication having a flow rate R1. The second medication can be a fluid medication. In some implementations, the second medication can be same as the first medication. The controller 108, which controls the infusion by the infusion device 110, can receive the parameters including the flow rate R1, flow rate R2, time T1 and time T2 from the user interface 106. The clinician inputs these parameters into the user interface 106. After T2, the infusion device 110 can provide a flow rate R1 of the first medication to the patient 104.

FIG. 3 is a diagram 300 illustrating a waveform of multiple boluses of fluid medication delivered by the infusion device 110 to the patient 104. The flow rate of each bolus can be different from the flow rate of other boluses. The time duration for each bolus can be different from the time duration of other boluses. In some implementations, there can be a requirement that the total volume of each bolus remains the same/constant while the flow rates and the time durations can vary. The time durations can vary based on physiological requirements of the patient 104. For example, in examples where the bolus is provided once in every breath cycle, the time duration of the boluses can vary because each breath can have a different time duration.

In some variations, the flow rate of one or more boluses (for example, all boluses) can be the same, and/or the time duration of one or more boluses (for example, all boluses) can be the same.

The clinician can input the values of parameters including flow rates (R1 of a first medication, R2 of a second fluid medication, R3 of the second fluid medication, and R4 of the second fluid medication) and the times (T1, T2, T3, T4, T5, and T6) on the user interface 106, which can further provide these parameters to the controller 108, which can further actuate the infusion device 110 that can provide the boluses. In some implementations, the second medication can be same as the first medication.

FIG. 4 is a diagram 400 illustrating an infusion pump 402, which can be an example of the medical device 102. The infusion pump 402 can also be referred to as an infusion module. The infusion pump 402 can provide one or more boluses of a fluid medication to a patient 104. The infusion pump 402 can include the user interface 106, the controller 108, and the infusion device 110. A clinician can use the user interface 106 to select or specify parameters for administering the fluid medication. These parameters can include a specification or selection of type of fluid medication, mode (for example, single bolus mode or multiple bolus mode) of provision of the fluid medication, flow rate corresponding to each bolus of fluid medication, duration corresponding to each bolus of fluid medication, time or event for initiation of each bolus of fluid medication, and the like. After the clinician inputs the parameters on the user interface 106, the user interface 106 can send the parameters to the controller 108. The controller 108 can then actuate the infusion device 110 to automatically provide the one or more boluses to the patient 104 in accordance with the parameters.

The infusion pump 402 can be one of a peristaltic pump, a syringe pump, and a patient controlled analgesic pump. The infusion pump 402 can be either a small pump or a large pump. The small pump can be small in size (for example, less than or equal to five inches×five inches×two inches), can weigh less (for example, less than or equal to twenty pounds), and can provide a small volume (for example, less than or equal to two milliliters) of bolus. The large pump can be large in size (for example, more than five inches×five inches×two inches), can weigh more (for example, more than twenty pounds), and can provide a large volume (for example, more than two milliliters) of bolus.

FIG. 5 is a diagram 500 illustrating a user interface 502 wirelessly connected with an infusion device 504 that can deliver one or more boluses to a patient 104. In some implementations, the user interface 502 can be same as the user interface 106, and the infusion device 504 can be same as the infusion device 110. The user interface 502 can be wirelessly connected to the infusion device 504 via a network 506. The network 506 can be one or more of: a local area network, a metropolitan area network, a wide area network, a bluetooth network, an infrared network, internet or any other network.

FIG. 6 is a diagram 600 illustrating a user interface 602 connected with an infusion device 604 that can deliver one or more boluses to a patient 104. In some implementations, the user interface 602 can be same as the user interface 106, and the infusion device 604 can be same as the infusion device 110. The user interface 502 can be wirelessly connected to the infusion device 504 via one or more wires 606.

FIG. 7 is a diagram 700 illustrating an example of a user interface 702. The user interface can be same as at least one of user interfaces 106, 404, 502 and 602. The user interface 702 can include buttons 704, 706, 708, 710, 712, and 714, and a graphical display window 716.

The button 704 can allow the clinician to power on or power off the user interface. The powering off of the user interface may not stop the scheduled delivery of one or more boluses by the infusion device. However, the user interface 702 can include other buttons that can be used to stop, pause, restart, and/or cancel the scheduled delivery of the one or more boluses.

The button 706 can allow the clinician to select a fluid medication for the bolus. The fluid medication can be one of a crystalloid solution and a colloid solution. When the clinician clicks on the button 706, the software application executing the user interface 702 displays a list of pre-populated fluid medications that may be available. The clinician can select one of the fluid medications in the list by clicking on the desired fluid medication.

The button 708 can allow the clinician to select a mode of delivery of the one or more boluses. The different modes can be (a) a single bolus, such as the bolus of diagram 200 and (b) multiple boluses, such as the boluses described with respect to diagram 300. When the clinician clicks on the button 708, the software application executing the user interface 702 displays a pre-populated list of modes. The clinician can select one of the modes by clicking on the desirable mode. Based on the selected mode, the software application executing the user interface 702 can provide selection options for the buttons 710, 712, and 714 to the clinician.

The button 710 can allow the clinician to select flow rates of each bolus from a pre-populated list. In some implementations, the clinician can provide desirable or recommended values of the flow rates that may not exist in the list. When the clinician selects a single bolus mode after clicking the button 708, the software application executing the user interface 702 allows the user to select or specify a single value of flow rate. When the clinician selects a multiple boluses mode after clicking the button 708, the software application executing the user interface 702 allows the user to select or specify multiple values of flow rate, one value for each bolus. The clinician can select a flow rate by clicking on the desirable flow rate, or can specify a new flow rate by inputting a new value of flow rate.

The button 712 can allow the clinician to select time durations of each bolus from a pre-populated list. In some implementations, the clinician can provide desirable or recommended values of the time durations that may not exist in the list. When the clinician selects a single bolus mode after clicking the button 708, the software application executing the user interface 702 can allow the user to select or specify a single value of time duration. When the clinician selects a multiple boluses mode after clicking the button 708, the software application executing the user interface 702 allows the user to select or specify multiple values of time duration, one value for each bolus. The clinician can select a time duration by clicking on the desirable time duration, or can specify a new time duration by inputting a new value of the time duration.

The button 714 can allow the clinician to select a time for the initiation of each bolus. When the clinician selects a single bolus mode after clicking the button 708, the software application executing the user interface 702 can allow the user to select or specify a single value of initiation time. When the clinician selects a multiple boluses mode after clicking the button 708, the software application executing the user interface 702 allows the user to select or specify multiple values of initiation time, one value for each bolus. The clinician can select an initiation time by clicking on the desirable time duration, or can specify a new initiation time by inputting a new value of the initiation time.

In some implementations, the button 714 can allow the clinician to select an event, detection of which can trigger the delivery of a bolus. One example of such an event can be blood pressure going below a first threshold, wherein the systolic pressure may drop below one hundred and ten, and diastolic pressure may drop below seventy-five. Another example of an event can be blood pressure going above a second threshold, wherein systolic pressure may exceed one hundred and thirty, and diastolic pressure may exceed eighty-five. A yet another example of an event can be the heart rate dropping below a third threshold, such as below fifty beats per minute for certain patients. A further example of an event can be the heart rate exceeding a fourth threshold, such as exceeding one hundred and twenty beats per minute for some specific patients. Another example of an event can be respiratory rate of an individual being erratic. A further example of an event can be the respiratory rate going below a fifth threshold, such as ten breaths per minute. A yet another example of an event can be the respiratory rate going above a sixth threshold, such as ten breaths per minute. One or more (each, in some implementations) of the first threshold, the second threshold, the third threshold, the fourth threshold, the fifth threshold, and the sixth threshold can be varied based on an age of the patient and a type of the patient (for example, human being, dog, or horse).

To measure blood pressure, the medical device (which incorporates the infusion device that delivers the bolus) can include a sphygmomanometer. To measure heart rate, the medical device (which incorporates the infusion device that delivers the bolus) can include a heart rate monitor. To measure the respiratory rate, the medical device (which incorporates the infusion device that delivers the bolus) can include at least one respiratory rate sensor. Further, the medical device (which incorporates the infusion device that delivers the bolus) can include other monitors or detectors to measure other physiological conditions associated with the listed events.

The clinician can select an initiation time by clicking on the required event. Then, the infusion device can provide the bolus to the patient 104 whenever the selected event is detected by the respective monitor in the medical device.

The graphical display 716 can display a waveform of the one or more boluses, and show the parameters (for example, type of fluid, mode, flow rates, and times) selected or specified by the clinician. The display of the waveform is advantageous, as such a display clearly shows errors such as those caused by incorrect typing so that the clinician can correct those errors. For example, if the clinician incorrectly types a particular time with an extra “0” in the end, the graphical window displays the erratic waveform, which the clinician can see and correct accordingly.

In one implementation, the user interface 702 can include a button, which when clicked and irrespective of the use of buttons 704, 706, 708, 710, 712, and/or 714, the infusion device to deliver one or more boluses with pre-programmed parameters. Such a button can be useful when patients with similar health problems need to be treated with similar medications.

Further, the user interface 702 can have other buttons and displays to facilitate other implementations described herein.

FIG. 8 is a flow diagram 800 illustrating an automatic delivery of one or more boluses to a patient 104 based on a prior selection or specification of bolus parameters by a clinician.

A user interface can display a list of types of fluid to be delivered to the patient 104. The user interface can receive, from a clinician at 802, an input characterizing a selection of a type of fluid to be delivered as a bolus.

The user interface can display a list of modes of bolus delivery. The user interface can then receive, from a clinician at 804, an input characterizing a selection of a mode of bolus delivery.

The user interface can then display a list of flow rates of each bolus delivery. The user interface can receive, from a clinician at 806, an input characterizing a selection or specification of flow rates.

The user interface can further display a list of durations corresponding to each bolus. The user interface can receive, from a clinician at 808, an input characterizing a selection or specification of durations for each of the one or more boluses.

The user interface can then display a list of initiation times corresponding to each bolus. In some variations, the user interface can request a user to provide values of the one or more initiation times. In response, the user interface can receive, from a clinician at 810, an input characterizing a selection or specification of the initiation times for each of the one or more boluses.

Further, the user interface can additionally or alternately display a list of events. The user interface can receive, from a clinician at 810, an input characterizing a selection of an event.

The receipt of various inputs on the user interface can occur in any order. Such an order is based on a selection or specification of parameters by the clinician. The selection or specification can be performed as per the desire of the clinician or as per procedural requirements.

A controller can connect, at 812, the infusion device with an appropriate storage container (for example, a drug storage bottle) from a plurality of available storage containers so that the fluid medication of only the connected storage container can be delivered to the patient 104. Such a connection can be made immediately after the clinician selects, at 802, the type of fluid for the medication. In one variation, this connection between the infusion device and the appropriate storage container can be made manually by the clinician.

The controller can activate, at 814, a delay timer that can detect the times T1, T2, and other times, if any. The delay timer can be connected to the infusion device either with a wire or wirelessly via a communication network. When an initiation time is detected, the timer can send an actuation signal to the controller. In some implementations, the controller can activate event monitors, which can detect events associated with detecting one or more of: blood pressure, heart rate, respiratory rate, and any other physiological event. When such an event is detected, the event monitor can send an actuation signal to the controller.

When the controller receives the actuation signal from the delay timer or the event monitor, the controller can actuate, at 816, the infusion device to provide a bolus to the patient 104. When the bolus is being provided, the delay timer can be again activated to keep a track of the duration of the bolus.

FIG. 9 is a system diagram 900 illustrating a computing landscape 901 that can include the user interface (106, 404, 502, 602, 702), the controller 108, the infusion device (110, 406, 504, 604) and the medical device 102 (which can be one of medical devices 916) within a healthcare environment, such as a hospital, a clinic, a laboratory, or any other environment. Various devices and systems, both local to the healthcare environment and remote from the healthcare environment, can interact via at least one computing network 902. This computing network 902 can provide any form or medium of digital communication connectivity (i.e., wired or wireless) amongst the various devices and systems. Examples of communication networks include a local area network (“LAN”), a wide area network (“WAN”), and the Internet. In some cases, one or more of the various devices and systems can interact directly via peer-to-peer coupling (either via a hardwired connection or via a wireless protocol such as Bluetooth or WiFi). In addition, in some variations, one or more of the devices and systems communicate via a cellular data network.

In particular, aspects of the computing landscape 100 can be implemented in a computing system that includes a back-end component (e.g., as a data server 904), or that includes a middleware component (e.g., an application server 906), or that includes a front-end component (e.g., a client computer 908 having a graphical user interface or a Web browser through which a user can interact with an implementation of the subject matter described herein), or any combination of such back-end, middleware, or front-end components. A client 908 and servers 904 and 906 are generally remote from each other and typically interact through the communications network 902. The relationship of the clients 908 and servers 904, 906 arises by virtue of computer programs running on the respective computers and having a client-server relationship to each other. Clients 908 can be any of a variety of computing platforms that include local applications for providing various functionalities within the healthcare environment. Example clients 908 include, but are not limited to, desktop computers, laptop computers, tablets, and other computers with touch-screen interfaces. The local applications can be self-contained in that they do not require network connectivity and/or they can interact with one or more of the servers 904, 906 (e.g., a web browser).

A variety of applications can be executed on the various devices and systems within the computing landscape such as electronic health record applications, medical device monitoring, operation, and maintenance applications, scheduling applications, billing applications and the like.

The network 902 can be coupled to one or more data storage systems 910. The data storage systems 910 can include databases providing physical data storage within the healthcare environment or within a dedicated facility. In addition, or in the alternative, the data storage systems 910 can include cloud-based systems providing remote storage of data in, for example, a multi-tenant computing environment. The data storage systems 910 can also comprise non-transitory computer readable media.

Mobile communications devices 912 can also form part of the computing landscape 100. The mobile communication devices 912 can communicate directly via the network 902 and/or they can communicate with the network 902 via an intermediate network such as a cellular data network 914. Various types of communication protocols can be used by the mobile communication devices 912 including, for example, messaging protocols such as SMS and MMS.

Various types of medical devices 916 can be used as part of the computing landscape 100. The medical devices 916 can include one or more of the medical device 102, the user interface 106, the controller 108, and the infusion device 110. These medical devices 916 can include, unless otherwise specified, any type of device or system with a communications interface that characterizes one or more physiological measurements of a patient and/or that characterize treatment of a patient. In some cases, the medical devices 916 communicate via peer to peer wired or wireless communications with another medical device 916 (as opposed to communicating with the network 902). For example, the medical device 916 can comprise a bedside vital signs monitor that is connected to other medical devices 916, namely a wireless pulse oximeter and to a wired blood pressure monitor. One or more operational parameters of the medical devices 916 can be locally controlled by a clinician, controlled via a clinician via the network 902, and/or they can be controlled by one or more of a server 904 and/or 906, a client 908, a mobile communication device 912, and/or another medical device 916.

The computing landscape 100 can provide various types of functionality as can be required within a healthcare environment such as a hospital. For example, a pharmacy can initiate a prescription via one of the client computers 908. This prescription can be stored in the data storage 910 and/or pushed out to other clients 908, a mobile communication device 912, and/or one or more of the medical devices 916. In addition, the medical devices 916 can provide data characterizing one or more physiological measurements of a patient and/or treatment of a patient (e.g., medical device 916 can be an infusion management system, etc.). The data generated by the medical devices 916 can be communicated to other medical devices 916, the servers 904 and 906, the clients 908, the mobile communication devices 912, and/or stored in the data storage systems 910.

Various implementations of the subject matter described herein can be realized/implemented in digital electronic circuitry, integrated circuitry, specially designed application specific integrated circuits (ASICs), computer hardware, firmware, software, and/or combinations thereof. These various implementations can be implemented in one or more computer programs. These computer programs can be executable and/or interpreted on a programmable system. The programmable system can include at least one programmable processor, which can be have a special purpose or a general purpose. The at least one programmable processor can be coupled to a storage system, at least one input device, and at least one output device. The at least one programmable processor can receive data and instructions from, and can transmit data and instructions to, the storage system, the at least one input device, and the at least one output device.

These computer programs (also known as programs, software, software applications or code) can include machine instructions for a programmable processor, and can be implemented in a high-level procedural and/or object-oriented programming language, and/or in assembly/machine language. As can be used herein, the term “machine-readable medium” can refer to any computer program product, apparatus and/or device (for example, magnetic discs, optical disks, memory, programmable logic devices (PLDs)) used to provide machine instructions and/or data to a programmable processor, including a machine-readable medium that can receive machine instructions as a machine-readable signal. The term “machine-readable signal” can refer to any signal used to provide machine instructions and/or data to a programmable processor.

To provide for interaction with a user, the subject matter described herein can be implemented on a computer that can display data to one or more users on a display device, such as a cathode ray tube (CRT) device, a liquid crystal display (LCD) monitor, a light emitting diode (LED) monitor, or any other display device. The computer can receive data from the one or more users via a keyboard, a mouse, a trackball, a joystick, or any other input device. To provide for interaction with the user, other devices can also be provided, such as devices operating based on user feedback, which can include sensory feedback, such as visual feedback, auditory feedback, tactile feedback, and any other feedback. The input from the user can be received in any form, such as acoustic input, speech input, tactile input, or any other input.

The subject matter described herein can be implemented in a computing system that can include at least one of a back-end component, a middleware component, a front-end component, and one or more combinations thereof. The back-end component can be a data server. The middleware component can be an application server. The front-end component can be a client computer having a graphical user interface or a web browser, through which a user can interact with an implementation of the subject matter described herein. The components of the system can be interconnected by any form or medium of digital data communication, such as a communication network. Examples of communication networks can include a local area network, a wide area network, internet, intranet, Bluetooth network, infrared network, or other networks.

The computing system can include clients and servers. A client and server can be generally remote from each other and can interact through a communication network. The relationship of client and server can arise by virtue of computer programs running on the respective computers and having a client-server relationship with each other.

Although a few variations have been described in detail above, other modifications can be possible. For example, the logic flows depicted in the accompanying figures and described herein do not require the particular order shown, or sequential order, to achieve desirable results. Other embodiments may be within the scope of the following claims.

What is claimed is:

1. A method comprising:
displaying, in a graphical user interface, a list of possible flow rates of one or more boluses of a fluid medication to be delivered to a patient and a list of possible durations for the one or more boluses, and a list of possible initiation times for the one or more boluses;
receiving, from the graphical user interface and by a controller connected to the graphical user interface, selections of one or more flow rates from the list of possible flow rates for the one or more boluses, one or more durations from the list of possible durations for the one or more boluses, and one or more initiation times from the list of possible initiation times for the one or more boluses, the selections being performed via the graphical user interface by a clinician; and
delivering, by an infusion device actuated by the controller based on the selections, the one or more boluses to the patient.

2. The method of claim 1, wherein the infusion device prior to the delivery of the one or more boluses infuses the fluid medication at a first rate and the infusion device immediately subsequent to the delivery of the one or more boluses automatically and without human intervention infuses the fluid medication at the first rate, the first rate being different than a flow rate for the one or more boluses.

3. The method of claim 1, further comprising:
displaying, in the graphical user interface, a list of possible types of fluid in the fluid medication; and
receiving, from the graphical user interface and by the controller, a selection of a type of fluid to be used in the fluid medication.

4. The method of claim 1, further comprising:
displaying, in the graphical user interface, a list of possible modes for delivery of the one or more boluses, the modes comprising a single bolus mode and a multiple bolus mode, the single bolus mode characterizing that a single bolus of fluid medication is to be delivered, the multiple bolus mode characterizing that two or more boluses are to be delivered; and
receiving, from the graphical user interface and by the controller, a selection of a mode to be used for delivering the one or more boluses.

5. The method of claim 1, further comprising:
displaying, in the graphical user interface, a list of possible initiation events for each bolus;
receiving, from the graphical user interface and by the controller, a selection of an initiation event; and
delivering, by the infusion device, at least one of the one or more boluses when the initiation event occurs.

6. The method of claim 5, wherein the initiation events comprise one or more of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

7. The method of claim 1, further comprising:
displaying, in the graphical user interface, a graphical waveform characterizing the selections of the one or more flow rates, the one or more durations, and the one or more initiation times.

8. The method of claim 1, further comprising:
generating, by an alarm device actuated by the controller, one or more alarms when a volume of a delivered bolus of the fluid medication either drops below a lower limit threshold or exceeds an upper limit threshold.

9. A system comprising:
a user interface device to display a list of possible flow rates of one or more boluses of a fluid medication to be delivered to a patient, a list of possible durations for the one or more boluses, and a list of possible initiation times for the one or more boluses, the user interface device receiving selections of one or more flow rates of corresponding one or more boluses, one or more durations of the corresponding one or more boluses, and one or more initiation times of the corresponding one or more boluses;
a controller to receive the selections from the user interface device; and
an infusion device to receive at least one control signal from the controller based on the selections, the at least one control signal actuating the infusion device to deliver the one or more boluses to the patient.

10. The system of claim 9, wherein the infusion device prior to the delivery of the one or more boluses infuses the fluid medication at a first rate and the infusion device immediately subsequent to the delivery of the one or more boluses automatically and without human intervention infuses the fluid medication at the first rate, the first rate being different than a flow rate for the one or more boluses.

11. The system of claim 9, wherein the user interface device:
displays a list of possible types of fluid in the fluid medication;
displays a list of possible modes for delivery of the one or more boluses, the modes including a single bolus mode and a multiple bolus mode, the single bolus mode characterizing that a single bolus of fluid medication is to be delivered, the multiple bolus mode characterizing that a plurality of boluses are to be delivered; and
displays a list of possible initiation events for each bolus.

12. The system of claim 11, wherein the user interface device:
receives a selection of a type of fluid to be used in the fluid medication;
receives a selection of a mode to be used for delivering the one or more boluses; and
receives a selection of an initiation event, the controller actuating the infusion device when the initiation event occurs so as to deliver at least one of the one or more boluses.

13. The system of claim 12, wherein the initiation event comprises at least one of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

14. The system of claim 12, wherein the user interface device comprises a graphical display area that displays a graphical waveform characterizing the selections of the one or more flow rates, the one or more durations, the one or more initiation times, the type of fluid, the mode, and the initiation event.

15. The system of claim 10, further comprising:
an alarm device that generates one or more alarms when a volume of a delivered bolus of the fluid medication either drops below a lower limit threshold or exceeds an upper limit threshold.

16. The system of claim 15, wherein the alarm comprises at least one of: a pop-up message displayed on the graphical user interface device, a loud sound, a short messaging service notification, an email, and a social network message.

17. A non-transitory computer program product storing instructions that, when executed by at least one programmable processor, cause the at least one programmable processor to perform operations comprising:
displaying, in a graphical user interface of a user interface device, a list of possible flow rates of a bolus of a fluid medication to be delivered to a patient and a list of possible durations for the bolus;
receiving, from the graphical user interface and by a controller connected to the graphical user interface, selections of a flow rate of the bolus from the list of possible flow rates, and a duration of the bolus from the list of possible durations, the selections being performed on the graphical user interface by a clinician; and
delivering, by an infusion device actuated by the controller and based on the selections, the bolus to the patient.

18. The computer program product of claim 17, wherein:
the infusion device infuses, prior to the delivery of the bolus, the fluid medication at a first rate, the first rate being different than a flow rate for the one or more boluses; and
the infusion device infuses, immediately subsequent to the delivery of the bolus automatically and without human intervention, the fluid medication at the first rate.

19. The computer program product of claim 17, wherein the operations further comprise:
displaying, in the graphical user interface, a list of possible types of fluid in the fluid medication; and
receiving, from the graphical user interface and by the controller, a selection of a type of fluid to be used in the fluid medication.

20. The computer program product of claim 17, wherein the operations further comprise:
displaying, in the graphical user interface, a list of possible initiation events for the bolus;
receiving, from the graphical user interface and by the controller, a selection of an initiation event; and
delivering, by the infusion device, the bolus when the initiation event occurs.

21. The computer program product of claim 21, wherein the initiation events comprise one or more of: decrease of blood pressure below a first threshold, increase of blood pressure above a second threshold, decrease of heart rate below a third threshold, increase of heart rate above a fourth threshold, decrease of respiratory rate below a fifth threshold, and increase of respiratory rate above a sixth threshold.

22. The computer program product of claim 17, wherein the operations further comprise:
displaying, in the graphical user interface, a graphical waveform characterizing the selections of the flow rate and the duration.

23. The computer program product of claim 17, wherein the operations further comprise:
generating, by an alarm device actuated by the controller, one or more alarms when a volume of a delivered bolus of the fluid medication either drops below a lower limit threshold or exceeds an upper limit threshold.
